(19) United States
(12) Patent Application Publication
(10) Pub. No.: US 2005/0026854 A1
(43) Pub. Date: Feb. 3, 2005

(54) Selective elimination of cd52and uses thereof

(76) Inventor: James William Young, Pelham Manor, NY (US)

Correspondence Address: Albert Wai Kit Chan;Law Offices of Albert Wai Kit Chan, World Plaza Suite 604, 141 07 20th Avenue, Whitestone, NY, 11357, US

(21) Appl. No.: 10/495,084

(22) PCT Filed: Nov. 1, 2002
(86) PCT No.: PCT/US02/35140

Related U.S. Application Data
(60) Provisional application No. 60/335,436, filed on Nov. 5, 2001.

(51) Int. Cl. A61K 48/00; A61K 39/395

(52) U.S. Cl. 514/44; 424/144.1

(57) ABSTRACT

This invention provides an agent capable of destruction of CD52⁺ cells, including CD52⁺ dendritic cells, without affecting CD52 negative dendritic cells. CD52 negative dendritic cells include hut are not limited to Langerhans cells (LCs) or dermal-interstitial dendritic cells (DDC-IDCs). This invention also provides an agent capable or selective elimination of CD52⁺ dendritic cells. This invention further provides an agent capable of protection of CD52 negative dendritic cells as well as a composition comprising the above described agent, a pharmaceutical composition comprising an effective amount of the above agent, and a pharmaceutically acceptable carrier. Finally, this invention provides various uses of the above agent and the above compositions.

This application claims priority of U.S. Ser. No. 60/335,436, filed 05 Nov. 2001, the content of which is incorporated by reference here into this application.

The invention disclosed herein was supported in part by National Cancer Institute, National Institutes of Health Grant Nos. P01 CA 23766, R01 CA 83070 and P01 CA 59350. Accordingly, the United States Government may have certain rights in this invention.

Throughout this invention, various references are cited. Disclosures of these publications in their entireties are hereby incorporated by reference into this application to describe more fully the state of the art to which this invention pertains.

### BACKGROUND OF THE INVENTION

CD52⁺ is a small, 12 aa, phosphatidyl inositol (GPI)-anchored membrane glycoprotein expressed by lymphocytes, especially T cells, as well as monocytes, macrophages, monocyte-derived dendritic cells (moDCs), and the epithelial cells of the distal epididymis and vas deferens (1,3). All of the functions of CD52⁺ may not yet be known, but it constitutes at least a target for complement-mediated cell lysis and antibody mediated cellular cytotoxicity (4,5). Complement activation, however, is neither necessary nor sufficient for MAb depletion of CD52⁺ cells in vivo (6,7). Antibody dependent cellular cytotoxicity (ADCC) is likely the more important mechanism for depleting CD52⁺ cells sensitized in vivo by approximately 1000 fold less antibody than required for complement-dependent lysis (8). These amounts of antibody approximate the levels detected in patients receiving alemtuzumab in vivo (9).

Alemtuzumab (CAMPATH-1H) is a recombinant DNA-derived, humanized MAb directed against CD52⁺ that is very efficient in mediating lymphocyte depletion both in vitro and in vivo (10). Treatment with humanized anti-CD52⁺ in vivo mitigates GvHD and promotes engraftment, even in adult recipients of unmodified allografts from unrelated and/or mismatched donors (11,12). This is especially noteworthy because other regimens that achieve comparable degrees of T cell depletion or immune suppression have not always proven similarly successful in these settings. This led to the question of whether anti-CD52⁺ targets different types of DCs in addition to depleting T cells.

DCs were studied because these are the most potent antigen-presenting cells and the most critical to initiation of cellular immune responses (13-15). A growing body of data has also emerged regarding the development of dendritic cells and their hematopoietic relationship to other leukocytes (16-25). From these studies one can distinguish at least three different types of myeloid DCs, for which investigators increasingly find different specialized functions. These myeloid DCs comprise at least CD34+ hematopoietic progenitor cell (HPC)-derived Langerhans cells (LCs) and dermal-interstitial dendritic cells (DDC-IDCs), as well as CD14+ blood monocyte-derived dendritic cells (moDCs) (16-25). Phenotypically, all three mature myeloid DC types are class II MHC^{bright}, CD86⁺⁺, CD14^{neg}, CD11c⁺, and importantly CD83⁺ (26). Langerhans cells are CD11b^{neg}, whereas the other two are CD11b⁺ (17,27).

CD14+ monocytes express abundant CD52⁺, as do monocyte-derived DCs identified by a monoclonal antibody (MAb) CMRF-56². Expression and function of the CD52⁺ antigen on all three myeloid DC types were evaluated, however. The CD52⁺ expression pattern of CD34⁺ HPC-derived, cytokine-generated LCs and DDC-IDCs, as well as their resident counterparts in skin and gut mucosa were especially studied. These were compared to the well-characterized moDCs generated in vitro, which have all the properties ascribed to and expected of DCs, but which have proven difficult to identify specifically in vivo.

The findings suggest additional mechanisms that may underlie the efficacy of anti-CD52⁺, which go beyond T cell-depletion. These results may also have important implications for future studies to determine whether deleterious graft-host interactions like GvHD can be distinguished at the level of antigen presentation from beneficial interactions like graft-versus-tumor activity and immune reconstitution.

### DETAILED DESCRIPTION OF THE FIGURES

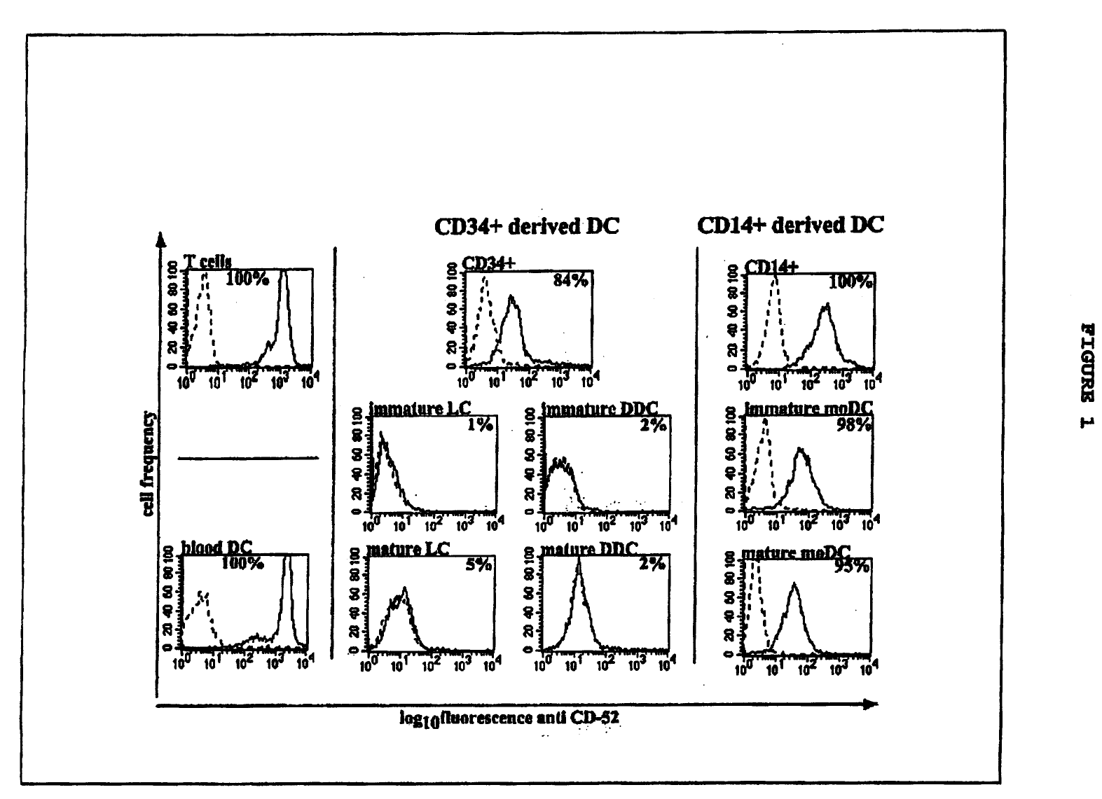

FIG. 1. Flow cytometry analysis of CD52⁺ expression by different myeloid DC populations and their precursors.

CD52 expression is limited to moDCs, and is never expressed by CD34+ HPC-derived LCs or DDC/IDCs. Flow cytometric analyses were performed to determine CD52⁺ surface expression by immature (surface CD83 neg, intracellular CD83+) and mature (CD83+) myeloid DCs and their precursors. T cells provided a positive control. Monocytes as well as their immature and mature moDC derivatives expressed abundant CD52⁺, although the mean fluorescent intensity (MFI) decreased somewhat with maturation. Circulating, immature DCs, defined as CD11c⁺, lineage (CD3, CD14, CD16, CD20)-negative cells gated from total PBMCs, also expressed CD52⁺ abundantly with an MFI comparable to that of T cells. A substantial proportion of CD34+ hematopoietic progenitor cells (HPCs) expressed CD52⁺, although at a relatively low MFI compared to the positive T cell control. CD11b^{neg} LCs and CD11b⁺ DDC-IDCs displayed no detectable expression of CD52⁺ above background, independent of the maturation state. Bold line histograms represent the reactivity of anti-CD52⁺ MAb with the selected population, and broken line histograms depict the isotype control. One experiment representative of three is shown.

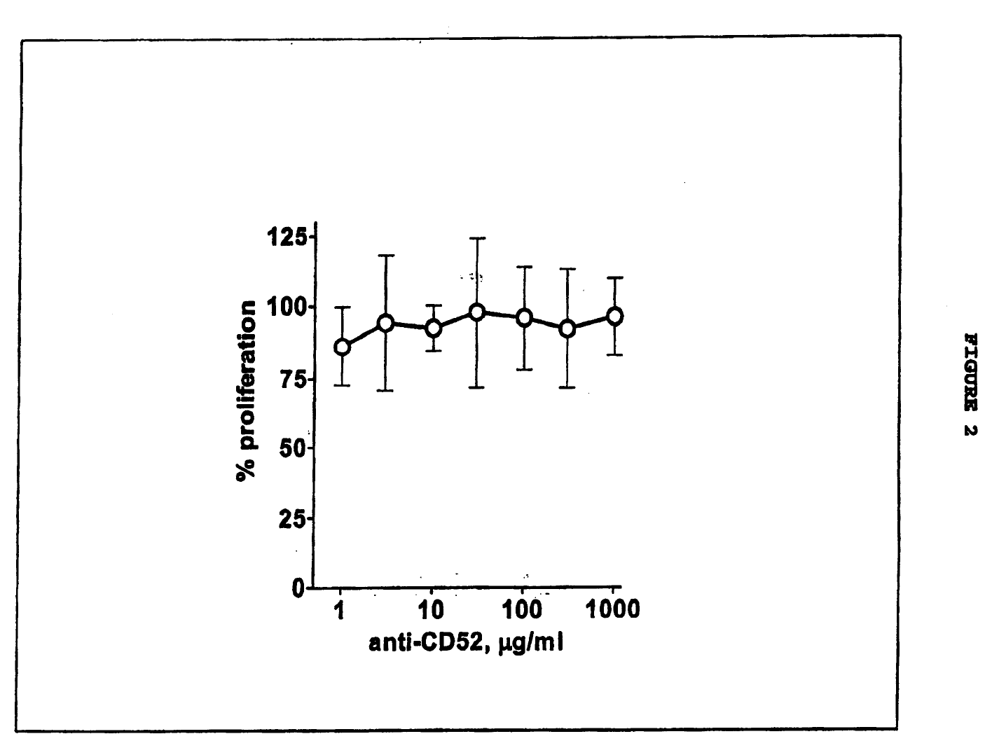

FIG. 2. Humanized anti-CD52⁺, in the absence of complement-mediated lysis, does not inhibit the immunostimulatory properties of DCs.

Anti-CD52 does not inhibit moDC stimulation of allogeneic T cells. Mature CD83⁺ moDCs were combined with purified allogeneic T cells at a fixed ratio of 1 DC:30 T cells in triplicate round-bottomed microwells. Humanized anti-CD52⁺ (alemtuzumab, CAMPATH-1H) was added to the alloMLR cultures in graded doses from 1 ug/ml to 1 mg/ml final concentration. Rituximab, a non-reactive humanized anti-CD20 MAb, was used as a negative control. Complete RPMI medium was supplemented with 10% heat-inactivated (complement-depleted) single donor normal human serum. T cell proliferation (3HTdR incorporation) in the anti-CD52⁺ condition was divided by that in the anti-CD20 condition to obtain a percent proliferation relative to normal, using anti-CD20 as a negative control MAb. Proliferation in the control anti-CD20 condition varied between 80,000 and 200,000 amongst six different allogeneic pairings, and the figure summarizes the averaged triplicate means +/−S.D. from the six separate experiments.

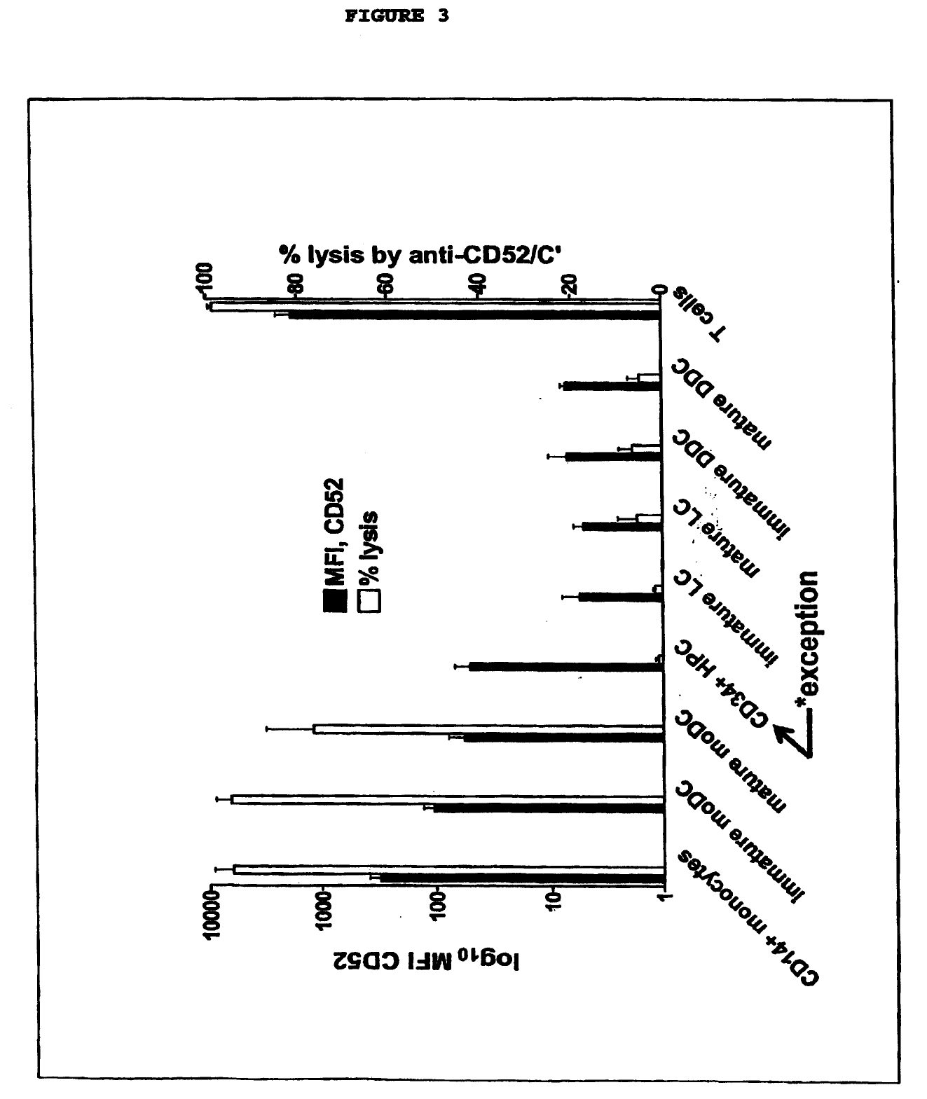

FIG. 3. Anti-CD52⁺/complement-dependent lysis is proportional to the surface density of the CD52⁺ epitope.

Susceptibility of anti-CD52/C′ lysis mirrors the amount of surface CD52 expression. DC subsets and their progenitors were compared for surface expression of CD52⁺ and sensitivity to complement-dependent lysis by humanized anti-CD52⁺. Filled bars indicate the MFI (mean fluorescent intensity) of CD52⁺ expression, plotted against the left Y-axis (note log (10) scale). Empty bars, plotted against the right Y axis, represent the percentage of cells lysed after opsonization with an excess of humanized anti-CD52⁺ (1 mg/ml alemtuzumab/CAMPATH-1H), thorough washing, and exposure either to 50% plasma without heat-inactivation or the equivalent of commercial complement. Complement exposure was normally one hour at 37° C., but cellular resistance to lysis was also confirmed even after overnight incubation. Expression of CD52⁺ and sensitivity to anti-CD52⁺/complement-mediated lysis in vitro are strongly correlated, with the exception of CD34+ HPCs (see also MFI depicted in FIG. 1). This graph summarizes three independent experiments, and error bars represent the SD of the triplicate means.

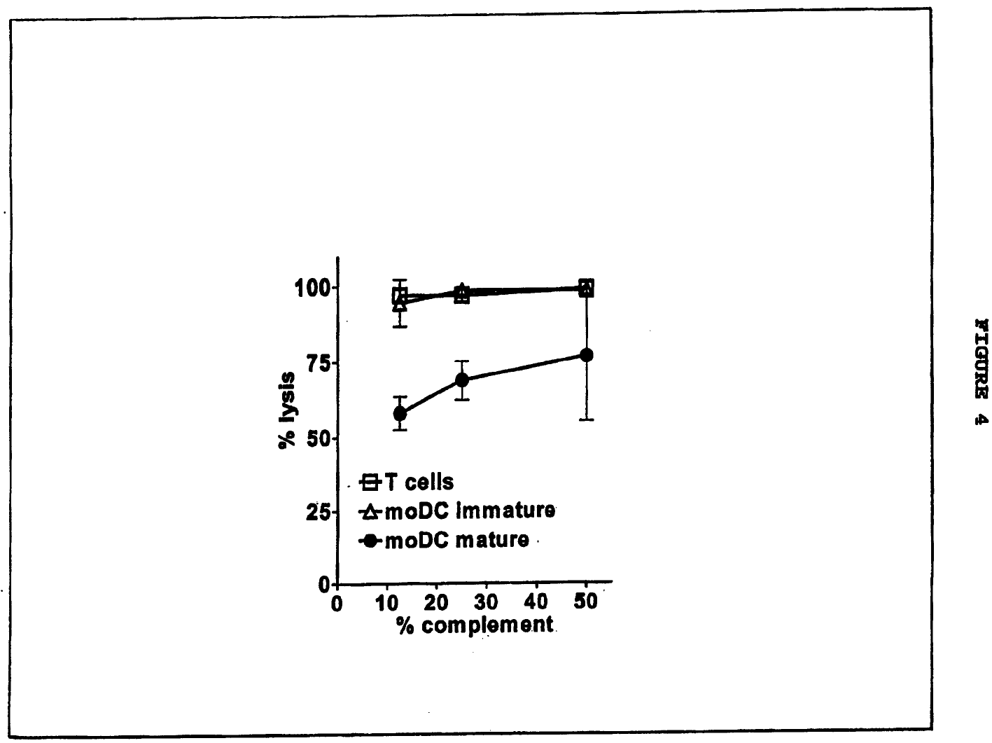

FIG. 4. Mature moDCs are less sensitive than monocyte precursors and immature moDCs to anti-CD52⁺/complement lysis, in proportion to their lower CD52⁺ expression.

Immature moDCs and T-cells are comparably sensitive to anti-CD52/C′ and more susceptible than mature moDCs. Cells were cultured in triplicate overnight in the presence of humanized anti-CD52⁺ (alemtuzumab, CAMPATH-1H) and complement. The source of complement was either fresh human plasma or commercial complement in an equivalent concentration, ranging from 50% to 12.5% plasma in the culture medium. Percent lysis was calculated based on the remaining viable cells that excluded trypan blue on a direct hemacytometer. count. Three experiments are summarized, and error bars represent the SD of the averaged triplicate means. MAb was used at 1 mg/ml in the experiments depicted, to confirm that resistant cells were insensitive even to anti-CD52⁺ used in excess. Pairwise comparison of 3 groups performed using the Wilcoxon rank sum statistic yielded a P-value between 0.2 and 0.33 for mature moDCs compared with immature moDCs or T cells in three concentrations of human plasma.

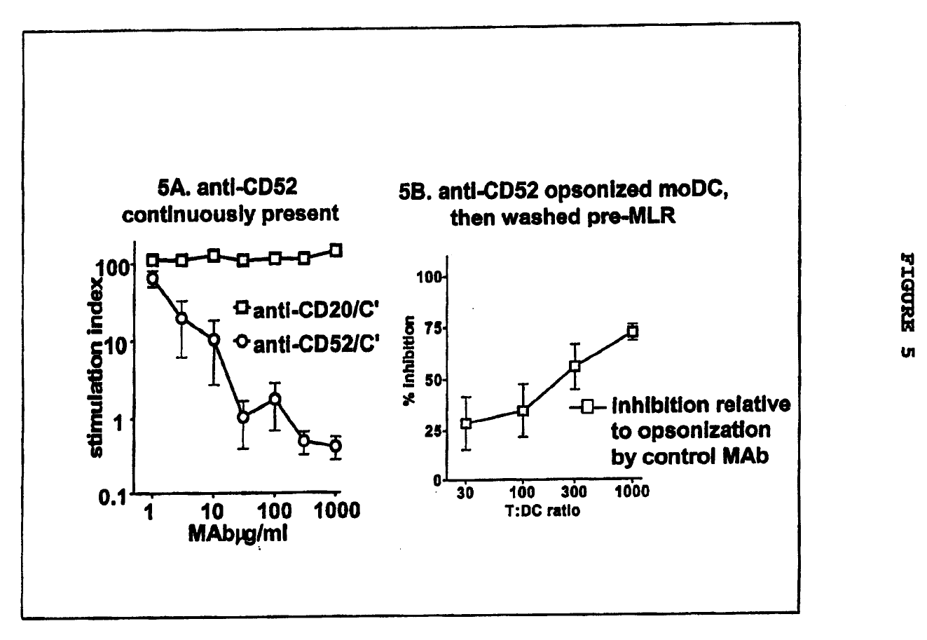

FIG. 5. Humanized anti-CD52⁺, in the presence of complement, alters moDC stimulation of allogeneic T cell proliferation in a dose dependent manner.

Anti-CD52 inhibits moDC allostimulatory activity in the presence of plasma or complement. 5A. Mature CD83+ moDCs were combined with allogeneic T cells at a fixed ratio of 1 DC:30 T cells in mixed leukocyte reactions (alloMLRs). Humanized anti-CD52⁺ (alemtuzumab, CAMPATH-1H) was added in graded doses from 1 ug/ml to 1 mg/ml. Complete RPMI was supplemented with complement-replete human plasma (not heat inactivated) or an equivalent amount of commercial complement. The proliferation of responder T cells in the continuous presence of humanized anti-CD52⁺/complement, divided by the proliferation in the presence of control humanized anti-CD20/complement, yielded a stimulation index at each dose of MAb. 100% control proliferation ranged from 50,000 to 200,000 cpm 3HTdR incorporation in three different allogeneic combinations. This inhibitory effect is due to MAb/complement-dependent lysis of both stimulator and responder cells, although the mature moDCs are relatively less sensitive so the predominant effect is likely T cell based. The graph summarizes three experiments, and the error bars represent the SD of the averaged triplicate means from each experiment. Comparison of these two groups based on Wilcoxon rank sum test showed p-values <0.02 for doses between 1 ug/ml to 1000 ug/ml.

5B. Immature moDCs were opsonized with an excess of humanized anti-CD52⁺ (alemtuzumab, 1 mg/ml final) or nonreactive control humanized anti-CD20 (rituximab, 1 mg/ml final) for 30-40 minutes on ice, thoroughly washed, and then exposed to complement or plasma (50% v/v) for one hour or overnight at 37° C. Both conditions started with the same number of cells and were handled identically. Yields and cell concentrations for reculture and terminal maturation of the moDCs, after MAb/complement treatment but before addition as stimulators to an allogeneic MLR, were based solely-on the control anti-CD20 condition. Already matured DCs proved too variable in their sensitivity to MAb/complement lysis to be used for these experiments. 3HTdR incorporation was assessed during the last 12 hrs of a 4-5 day culture. The percent inhibition at each T:DC dose was calculated as follows: 100−{(mean cpm of T cells stimulated by anti-CD52⁺ treated moDCs/mean cpm of T cells stimulated by anti-CD20 treated moDCs)×100}. Shown are the results of three independent experiments and the SD of the mean percent inhibition at each T:DC dose.

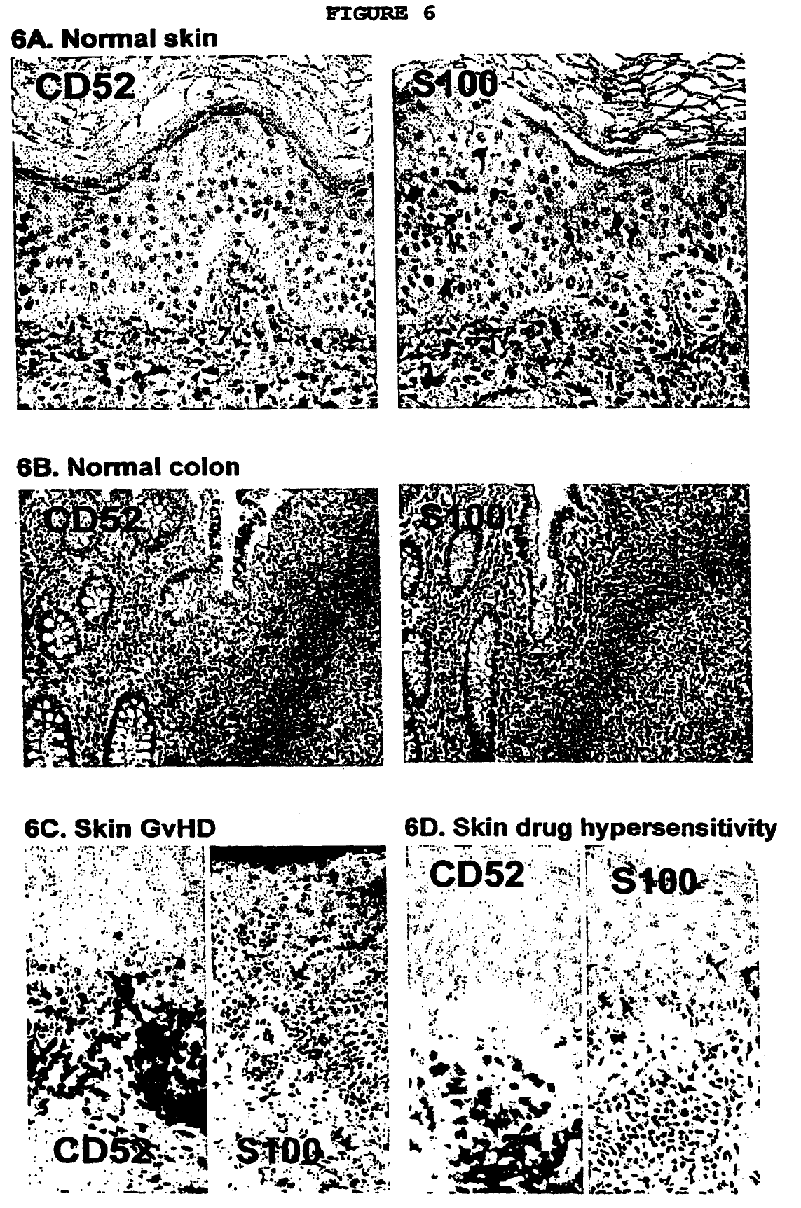

FIG. 6. Cutaneous or mucosal dendritic cells do not express CD52⁺ in vivo.

Resident mucocutaneous DCs do not express CD52. (6A) Normal skin: Intraepidermal dendritic cells (Langerhans cells) are immunopositive for S-100 protein but negative for CD52⁺. Apart from dermal inflammatory CD52⁺ T cell aggregates that could be seen as an artifact of the punch biopsy in some sections (not shown), no CD52⁺ dendritic profiles were seen in the dermis. Factor XIIIa staining confirmed the presence of dermal DCs (not shown). (6B) Normal colon: CD52⁺ T-lymphocytes surround a lymphoid follicle in a colonic biopsy. Mucosal dendritic cells, which are immunopositive for S-100 protein, are negative for CD52⁺. Germinal center B cells stained in this paraffin embedded and fixed tissue were also negative as expected. (6C) Graft-versus-host disease (GvHD) affecting the skin: Intraepidermal and dermal dendritic cells are immunopositive for S-100 protein, but negative for CD52⁺. Inflammatory T lymphocytes that are destroying the dermal-epidermal interface as part of the GvH reaction are CD52⁺, but there are no CD52⁺ dendritic profiles. (6D) Drug hypersensitivity reaction in the skin: CD52⁺ lymphocytes are seen in the superficial dermis. S-100 protein-positive dendritic cells are present in the epidermis and superficial dermis but are consistently negative for CD52⁺.

### DETAILED DESCRIPTION OF THE INVENTION

This invention provides an agent capable of destruction of CD52⁺ cells, including CD52⁺ dendritic cells, without affecting CD52 negative dendritic cells. CD52 negative dendritic cells include but are not limited to Langerhans cells (LCs) or dermal-interstitial dendritic cells (DDC-IDCs). This invention also provides an agent capable of selective elimination of CD52⁺ dendritic cells. This invention further provides an agent capable of protection of CD52 negative dendritic cells as well as a composition comprising the above described agent, a pharmaceutical composition comprising an effective amount of the above agent, and a pharmaceutically acceptable carrier. Finally, this invention provides various uses of the above agent and the above compositions.

As encompassed by this invention, the agent is capable of binding to CD52⁺ cells. In an embodiment, the agent is an antibody or portion thereof. As CD52⁺ is a known antigen, it would be within the skill of an ordinary artisan to produce antibodies capable of binding to CD52⁺. Either monoclonal or polyclonal antibodies may be produced. See Using Antibodies: A Laboratory Manual: Portable Protocol No. 1, by Ed Harlow and David Lane (1998).

This invention also provides for agents other than CAMPATH-1, which is the antibody that binds to and is capable of selective elimination of CD52⁺ dendritic cells. Antibodies that bind to CD52⁺, like CAMPATH-1, are known in the field. See U.S. Pat. No. 5,494,999, U.S. Pat. No. 5,846,534, U.S. Pat. No. 6,120,766, EPO 328,404, and WO 9,307,899 as examples. It is not the intention of this invention to cover a known agent. However, it is the intention of this invention to encompass compositions that comprise this known agent in effective amounts that are capable of selective elimination of CD52⁺ cells. In an embodiment the CD52⁺ cells include but are not necessarily limited to CD52⁺ dendritic cells. In a separate embodiment, the agent comprises an anti-sense nucleic acid molecule. This agent may further comprise a toxin. In accordance with this invention, the agent may include a suicide gene which is specifically targeted to CD52⁺ dendritic cells. The suicide gene approach is well-known in the art and was summarized by Woo, et al. (2000; U.S. Pat. No. 6,066,624) and Tiraby, et al. (1999; U.S. Pat. No. 5,856,153).

For the purposes of this invention, a “pharmaceutically acceptable carrier” means any of the standard pharmaceutical carriers. Examples of suitable carriers are well known in the art and may include but are not limited to any of the standard pharmaceutical carriers like phosphate buffered saline solutions, phosphate buffered saline containing Polysorb 80, water, emulsions such as oil/water emulsion, and various types of wetting agents. Other carriers may also include sterile solutions, tablets, coated tablets, and capsules.

Typically such carriers contain excipients like starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

This invention provides a pharmaceutical composition for prevention, treatment, or elimination of graft versus host disease comprising an effective amount of the above agent and a pharmaceutically acceptable carrier. This invention provides a pharmaceutical composition that promotes or maintains the graft-versus-tumor effect of hematopoietic stem cell transplantation, comprising an effective amount of the above agent and a pharmaceutically acceptable carrier.

This invention provides a method for preventing or treating graft versus host disease in a subject comprising selective elimination of CD52⁺ dendritic cells in the subject without affecting CD52 negative dendritic cells.

Graft-versus-host disease, GvHD, is an immunological disorder that limits the success and availability of allogeneic bone barrow or stem cell transplantation for treating some forms of otherwise incurable malignancies or hematologic or immunologic disorders. GvHD is a systemic inflammatory reaction that causes chronic illness, and GvHD may lead to death of the host mammal. At present, allogeneic transplants invariably run a substantive risk of associated GvHD, even where the donor has a high degree of histocompatibility with the host.

Donor T-cells reacting against systemically distributed incompatible host antigens may cause powerful inflammation manifested as GvHD. In GvHD, mature donor T-cells that recognize differences between donor and host become activated. To prevent and treat GvHD may involve administration of drugs like cyclosporin-A and corticosteroids. These treatments are expensive, have serious side effects, and must be given for prolonged periods of time. T-cell depletion that has used to prevent GvHD also requires sophisticated and expensive facilities and expertise. Too great a degree of T-cell depletion may lead to serious problems of hematopoietic graft failure, failure of immune reconstitution, infections, or relapse. Limited T-cell depletion leaves behind cells that are still competent to initiate GvHD. As a result, current methods of preventing or treating GvHD are only successful in limited donor and host combinations or at the expense of side effects that can range from mild to severe and life-threating. Many patients, therfore cannot be offered these life-saving treatments for which they might otherwise be elegible.

This invention provides a pharmaceutical composition comprising an amount of CD52 negative dendritic cells effective for hematopoietic stem cell transplantation and a pharmaceutically acceptable carrier. As used herein, the carrier can be physiologically saline or other media where the cells can be maintained. The CD52 negative dendritic cells effective for hematopoietic stem cell transplantation are capable of promotion or maintenance of allograft-versus-tumor activity without increasing the risk or incidence of graft versus host disease.

In accordance with this invention, CD52 negative dendritic cells may be harvested in a subject and propagated in vitro. Autologous or allogeneic treatments may be administered to patients who are in need of the CD52 negative dendritic cells.

This invention provides a method for preventing or treating graft versus host disease, in a subject comprising administration of an effective. amount of the above agent to the subject. This invention provides a method for prognosticating success of transplantation in a subject comprising steps of detecting the number of CD52 negative and/or positive dendritic cells before and after transplantation, wherein the decrease of the CD52⁺ dendritic cells and the relatively unchanged number of the CD52 negative dendritic cells, when compared before and after transplantation, indicates a high probability of success of the transplantation.

As used herein, transplantation includes but is not limited to hematopoietic stem cell and solid organ transplantation. One theme of this invention is to deplete CD52⁺ dendritic cells selectively in a subject. As an ordinary skilled artisan in this field can easily appreciate, selective depletion may also prove useful in treating some rare diseases, e.g. histiocytosis X.

This invention provides a kit comprising a compartment containing an agent capable of eliminating CD52⁺ positive dendritic cells without affecting CD52 negative dendritic cells.

This invention further provides the above kit that further comprises an agent capable of protecting CD52 negative dendritic cells.

This invention provides a kit comprising a compartment containing an agent capable of protecting CD52 negative dendritic cells.

This invention provides a kit comprising a compartment containing an amount of CD52 negative dendritic cells effective for promotion or maintenance of allograft-versus-tumor activity without increasing the risk or incidence of graft versus host disease.

This invention will be better understood from the examples that follow. However, one skilled in the art will readily appreciate that the specific methods and results discussed are merely illustrative of the invention as described more fully in the claims that follow thereafter.

### EXPERIMENTAL DETAILS

### Materials and Methods

Media and Reagents

RPMI 1640, 10 mM Hepes, 1% penicillin/streptomycin (Media Lab Core Facility, MSKCC), 50 uM 2-ME (GibcoBRL Life Technologies, Carlsbad, Calif.), 1% L-glutamine (GibcoBRL), and 1% heat-inactivated, autologous human plasma or serum were used for culture of moDCs with cytokines as outlined below. X-VIVO 15 (BioWhittaker, Walkersville, Md.), without either serum or plasma but supplemented with cytokines specified below, was used to culture CD34+ HPCs for the generation of LCs or DDC-IDCs. The DDC-IDC cultures, however, included an initial 5 days in IMDM, 1% penicillin/streptomycin (Media Lab, MSKCC), 1% L-glutamine (GibcoBRL), and 50 uM 2-ME (GibcoBRL), supplemented with 20% autologous plasma before transfer to X-VIVO 15. Allogeneic mixed leukocyte reactions (MLRs) were cultured in complete RPMI medium as above, supplemented with 10% heat-inactivated single, donor human serum but no exogenous cytokines.

Fresh human plasma (50% v/v) that was not heat-inactivated or commercial human complement (# S1764, Sigma, St. Louis, Mo.) was used for MAb/complement-dependent lysis. The commercial product, used strictly according to manufacturer's instructions, proved more stable and reproducible.

Recombinant human cytokines used for in vitro generation of DCs were GM-CSF (Immunex, Seattle, Wash.); IL-4, TNFα, TGFβ, c-kit-ligand or stem cell factor, FLT-3 ligand, IL-1β, IL-6 (all R&D Systems, Minneapolis, Minn.); and PGE2 (Calbiochem, San Diego, Calif.). Humanized anti-CD52⁺ was obtained as the pharmaceutical alemtuzumab (CAMPATH-1H; Berlex Laboratories, Richmond, Calif.) for functional studies in vitro. Humanized anti-CD20 was obtained as the pharmaceutical rituximab (Genentech, South San Francisco, Calif.) for functional studies in vitro as a control humanized MAb that would be nonreactive with T cells and myeloid DCs. Doses are specified for the respective cultures below.

Cell Purification and Generation of Dendritic Cells

All cells were obtained from healthy donors, most of whom were already serving as donors for allogeneic hematopoietic stem cell transplants (HSCT) either by harvesting bone marrow or pheresing G-CSF stimulated peripheral blood stem cells (PBSC). Donors signed informed consents for research sample collection protocols reviewed by the local Institutional Review Board.

MoDC precursors were obtained from tissue culture plastic-adherent peripheral blood mononuclear cells (PBMCs) after standard separation over Ficoll-Paque PLUS (#17-440-03, Amersham Pharmacia Biotech AB, Uppsala, Sweden). Tissue culture plastic-adherent-mononuclear cells were cultured in GM-CSF (1000 IU/ml) and IL-4.(500 IU/ml) as published²⁸. Medium and cytokines were replenished every 2 days. At approximately day 6, large FSC, HLA-DR+ cells expressed intracellular CD83, confirming commitment to DC differentiation, but very little cell surface CD83 (not shown). An inflammatory cytokine cocktail was added to these immature moDCs for terminal maturation and activation. This mixture comprised IL-1β (2 ng/ml), IL-6 (1000 IU/ml), TNFα (10 ng/ml) and PGE2 (5 mM/ml) 29,30. By day 8 of culture, large forward scatter, HLA-DR bright cells were CD14 negative and >90% surface CD83 and CD86 positive.

CD34+ HPCs were obtained by positive immunomagnetic selection from bone marrow or GCSF-elicited peripheral blood mononuclear cells separated over Ficoll-Paque PLUS (Amersham Pharmacia Biotech AB) according to manufacturer's instructions (CD34+ isolation kit and LS separation columns, Miltenyi Biotec, Bergisch Gladbach, Germany). LCs and DDC-IDCs were separately generated from CD34+ HPCs in the media described above and as previously published (17,18,20,22,23) (and Baggers et al., in preparation). Specific cytokine supplements included GM-CSF (1000 IU/ml), TNFα (5 ng/ml), c-kit-ligand (20 ng/ml), and FLT-3 ligand (50 ug/ml), with removal of c-kit-ligand and FLT-3 ligand from day 5-6 onward. CD34+ HPC cultures were replenished with cytokines and media on day 3, and thereafter approximately every other day.

Two amendments were made to these cytokine mixtures. For the specific generation of LCs, TGFβ-1 (10 ng/ml) was provided throughout the entire culture period (22,23). For the specific generation of DDC-IDCs, IL-4 (500 IU/ml) was added to suppress macrophage differentiation (31) when the cells were recultured at day 5-6 in X-VIVO 15 supplemented with GM-CSF and TNFα but without c-kit-ligand and FLT-3 ligand. By day 12, large FSC, HLA-DR+ cells- expressed intracellular CD83, confirming-commitment to DC differentiation, but little surface CD83. Terminal maturation of these immature LCs or DDC/IDCs was accomplished from day 12 to 14 of culture, by providing the same inflammatory cytokine cocktail used for moDCs above. The resultant large forward scatter, HLA-DR bright cells were CD14 negative, >60-70% CD83+, and approximately 90% CD86. LCs were CD11b neg, and DDC-IDCs were CD11b+. The remaining progeny in the total cell population were immature granulocytic cells, especially eosinophils, as intermediate steps to enrich for DCs and deplete other myeloid CD34+ HPC progeny were not undertaken.

T cells were obtained from the PBMC fraction that was nonadherent to tissue culture plastic. Nonadherence and elution from nylon wool columns (Polysciences, Warrington, Pa.) further purified these cells in excess of 95%.

Phenotypic Analyses by Flow Cytometry

Direct fluorescein (FITC) and phycoerythrin (PE) conjugated mouse anti-human MAbs included anti-CD3-FITC, anti-CD3-PE, anti-CD14-PE, anti-CD16-PE, anti-CD20-PE, anti-CD11b-PE, anti-CD11c-PE (Pharmingen, Franklin Lakes, N.J.); anti-CD4-PE, anti-CD8-PE, anti-CD14-PE, anti-CD14-FITC, anti-CD34-FITC, anti-CD11c-APC (Becton Dickinson, Franklin Lakes, N.J.); and anti-CD34-PE, anti-CD83-PE (Immunotech, Marseille, France). Isotype controls included IgG1-FITC, IgG1-PE, and IgG2a-FITC (Dako, Carpenteria, Calif.); and rat IgG2b-FITC (Serotec, Oxford, UK). Rat anti-human CD52⁺-FITC and its respective rat IgG2b-FITC control (Serotec, Oxford, UK) were used for phenotypic but not functional assessments of CD52⁺. Annexin-V (Early Apoptosis Detection Kit, Kamiya Biomedical Company, Seattle, Wash.) and propidium iodide staining respectively distinguished apoptotic and necrotic cells. Cytofluorographic evaluation used a FACScan (Becton-Dickinson, Immunocytometry Systems, San Jose, Calif.), gating for live events. For analysis of specific epitope, expression by DCs, candidate cells were first gated for large FSC, HLA-DR bright cells, after which 10,000 events were collected for analysis.

Monoclonal Antibody and Complement-Dependent Cell Lysis

MAb/complement-dependent lysis was used in vitro as a surrogate measurement of anti-CD52⁺ function in vivo. Assessment by ADCC in vitro, which is the more likely mechanism through which anti-CD52⁺ exerts its effects in vivo (6), proved not to be logistically feasible.

Cells were cultured in complete RPMI supplemented with 50% fresh human plasma without heat inactivation or with commercial complement equivalent to 50% plasma. As negative controls for complement, heat-inactivated (56° C. for 30min) normal human plasma or serum was used. Humanized anti-CD52⁺ (alemtuzumab, CAMPATH-1H) and nonreactive control humanized anti-CD20 (rituximab) were added at the concentrations indicated for each experiment. Cells were opsonized with MAb for 30-40 minutes on ice, thoroughly washed, and exposed to complement or plasma (50% v/v) for one hour. When certain DCs proved resistant to MAb/complement-dependent lysis, owing to their lack of CD52⁺ expression, complement exposure of MAb-opsonized cells was extended overnight to exclude the possibility that low level CD52⁺ expression might mediate some degree of cell targeting and lysis by alemtuzumab. After washing, the remaining viable cells were counted directly by Trypan blue exclusion on a hemacytometer.

Immunohistochemistry

Immunohistochemical studies were performed on formalin-fixed and paraffin-embedded tissues. The antibodies used included anti-S-100 protein (1:50,000;; Biogenix, San Ramon, Calif.) and anti-CD52⁺ (1:40; Serotec, Oxford, UK). The tissue sections were exposed to the antibodies in citrate buffer solution at pH6.0. Detection of the primary antibody was performed with a biotinylated secondary antibody (1:100; Vector, Burlingame, Calif.) for 30 min followed by an avidin-biotin complex system (Vector, Burlingame, Calif.), using diaminobenzidine tetrahydrochloride (DAB, Biogenix, San Ramon, Calif.) as chromogen. The slides were counterstained with Mayer's hematoxylin (Sigma, St. Louis, Mo.)

Mixed Leukocyte Reactions

DCs were cocultured with 10(5) purified allogeneic T cells (alloMLRs) in triplicate round-bottomed microwells at either a constant ratio of 30:1::T:DCs and variable concentrations of MAbs, or at variable T:DC ratios of 30:1 to 1000:1 in the presence of a constant MAb concentration. Medium for allogeneic MLRs consisted of complete RPMI supplemented with 10% single donor serum or plasma as above, but with no exogenous cytokines. DCs were extensively washed to remove cytokines before adding to T cells.

As only moDCs expressed CD52⁺ in appreciable amounts (see below), only moDCs were evaluated in allogeneic MLRs after treatment with anti-CD52⁺. The allogeneic MLRs were cultured in the continuous presence of alemtuzumab, which targeted both moDC stimulators and T cell responders. Alternatively, the moDCs were pretreated with humanized anti-CD52⁺ (alemtuzumab) or control humanized anti-CD20 (rituximab) and complement, thoroughly washed and then added to allogeneic stimulators in doses based on the DC yield in the control condition.

Proliferating T cells incorporated 3HTdR (1 uCi/well; New England Nuclear, Boston, Mass.) during the last 12 hrs of a 4-5 day culture. The amount of 3HTdR incorporated was measured in a beta scintillation: counter (Betaplate, Wallac, Perkin Elmer Life Sciences, Wellesley, Mass.).

Results

CD52⁺ is differentially expressed on dendritic cell subsets and. their precursors.

CD52⁺ expression was determined by flow cytometry using a FITC-conjugated rat anti-human CD52⁺ compared with a rat IgG2b-FITC control (FIG. 1 and Table 1). Purified T cells were included as positive controls given their known expression of CD52⁺. Fresh PBMCs were also stained and gated for candidate circulating, immature DCs, as CD11c+, lineage-negative (CD3, CD14, CD16, CD20 -negative) cells, from which determined that all expressed CD52⁺. Monocytes also expressed abundant amounts of CD52⁺, as expected; and more than 95% of the differentiated immature and mature moDCs expressed CD52⁺ as well. The mean fluorescent intensity (MFI) decreased somewhat with maturation, indicating a decrease in CD52⁺ density on the cell surface.

Approximately 80% of the starting CD34+ HPCs expressed CD52⁺, although at a lower surface density than PBMCs and moDCs based on the MFI. Most importantly, however, neither immature nor mature LCs or DDC-IDCs derived from these CD34+ HPCs in vitro ever expressed CD52⁺ at any subsequent stage of differentiation or maturation. This was confirmed by serial phenotyping from approximately day 3 until the end of the two week culture.

CD52⁺ has no proliferative or adhesive function in dendritic cell—T lymphocyte interactions.

To evaluate whether CD52⁺ alone influences DC function, allogeneic T cells and DCs were cocultured in the presence of humanized anti-CD52⁺ (alemtuzumab, CAMPATH-1H) compared with the nonreactive humanized anti-CD20 (rituximab) control 10% v heat-inactivated plasma or serum was used to avoid the introduction of complement. The more potent immunostimulatory, mature CD83+ moDCs were combined in a fixed ratio with allogeneic T cells (1 DC 30 T cells), and the dose of MAb added to the cultures was varied from 1 ug/ml to 1 mg/ml final. Humanized anti-CD52⁺ did not inhibit the formation of DC:T cell clusters, from which reactive T cell blasts emerge, as assessed by direct inspection using inverse phase microscopy (not shown). T cell proliferation also remained comparable to control conditions at all MAb doses (FIG. 2). Hence, conclude that CD52⁺ plays no role in adhesion or proliferation in allogeneic DC-T cell interactions.

Alemtuzumab (anti-CD52⁺, CAMPATH 1-H) does not impair development and maturation of moDC.

MAbs have several mechanisms by which they may exert their cellular effects. In addition to complement-mediated and ADCC, these mechanisms may also include the induction of apoptosis and the inhibition of metabolically active proteins like cytokines and growth factors (10,32). To evaluate any role of anti-CD52⁺ in this respect, humanized anti-CD52⁺ (alemtuzumab, CAMPATH-1H) was added at a maximum dose of 1 mg/ml during the generation of moDCs from CD14⁺ monocytes in 1% heat-inactivated autologous plasma with cytokines as above. Cell counts as well as flow cytometric analysis for apoptosis and maturation did not reveal any differences between anti-CD52⁺ supplemented cultures compared to controls (Table 2). Anti-CD52⁺ therefore causes neither apoptosis nor inhibition of DC development and maturation from CD52⁺ CD14+ class II MHC+ monocyte precursors.

Humanized anti-CD52⁺ induces complement-mediated lysis in proportion to the level of CD52⁺ expression.

MAb/complement-dependent lysis was used in vitro as a surrogate measurement of anti-CD52⁺ function in vivo, which is likely more dependent on ADCC(6). Cells were treated with anti-CD52⁺/complement or control anti-CD20/complement as described, and percent lysis was calculated based on the recovery of viable cells detected by Trypan blue exclusion on a direct hemacytometer count. Humanized anti-CD52⁺ lysed CD52⁺ cells in the presence of complement and in proportion to the level of surface CD52⁺ expression, with the exception of CD34+ HPCs (FIG. 3).

As expected, T cells were lysed very efficiently, almost up to 100%. CD14⁺ monocytes, immature moDCs, and mature moDCs also proved sensitive to complement-mediated lysis by humanized anti-CD52⁺. LCs and DDC-IDCs, however, proved resistant regardless of their maturation state, in keeping with their lack of CD52⁺ expression. The only CD52⁺ cells that were resistant to MAb/complement-dependent lysis were CD34+ HPCs, which had a lower surface density of CD52⁺ based on mean fluorescent intensity.

Although moDCs expressed abundant CD52⁺, independent of the maturation state, the MFI decreased somewhat with maturation. Mature moDCs proved less sensitive to anti-CD52⁺/complement-mediated lysis than immature moDCs and with much greater variation at the highest dose of complement. Reduced concentrations of complement to 12.5% resulted in diminished lysis and increased survival of approximately 40% of mature moDCs (FIG. 4). In contrast, MAb/complement-dependent lysis of immature moDCs and T cells remained complete at all concentrations of complement (FIG. 4). MAb was used at 1 mg/ml in the experiments depicted, to confirm that resistant cells were insensitive even to anti-CD52⁺ used in excess.

Humanized anti-CD52⁺/complement alters moDC stimulation of allogeneic T cell proliferation in a dose dependent manner.

The functional consequences of anti-CD52⁺ targeting of CD52⁺ cells in the context of a fully allogeneic MLR were evaluated. Cells were exposed continuously throughout the allogeneic MLR culture period to humanized anti-CD52⁺ alemtuzumab)/complement versus control humanized anti-CD20 (rituximab)/complement. Mature moDCs were used in these experiments as the optimal stimulators, even though they were more resistant to MAb/complement lysis (FIG. 3) than their less immunostimulatory, day 5-6 immature moDC precursors. The dominant effect, as shown in FIG. 5A, may therefore have been directed at the T cell responders. By holding constant the amount of complement, there was a wide range of inferred lytic activity, especially at MAb doses below 30 mcg/ml, which correspond to the levels recoverable from patients treated in vivo (9).

Alternatively, day 5-6 immature moDCs were opsonized with anti-CD52⁺, washed, and exposed to complement before subsequent cytokine-induced maturation and addition to allogeneic T cells in the MLR. Immature moDCs that were matured after MAb/complement exposure were used, because already matured moDCs do not exhibit consistent susceptibility to humanized anti-CD52⁺ (alemtuzumab)/complement. Pretreatment of the moDCs also spared T cells from the effects of the MAb and instead specifically targeted the moDCs. As shown in FIG. 5B, the percent inhibition relative-to control humanized anti-CD20 (rituximab) was most pronounced at the lower stimulator doses, likely due to the strength of an allogeneic stimulus when higher moDC doses were used. In any case these lower stimulator doses would approximate T:DC ratios expected in vivo, even under inflammatory conditions.

LCs and DDC/IDCs were not evaluated in these experiments, owing to their lack of CD52⁺ expression and their resistance to anti-CD52⁺/complement-dependent lysis. In lieu of exposing MAb-treated cells to complement for the experiments depicted in FIG. 5B, opsonized immature moDCs were with humanized anti-CD52⁺ (alemtuzumab) or control humanized anti-CD20 (rituximab), then cultured were with nonadherent autologous PBMCs dosed to provide an NK cell (CD3 negative, CD56+) to moDC ratio of 30:1 to 50:1. Use of the resultant cells (1500r 137 Cs) as allogeneic MLR stimulators yielded very inconsistent results. Further attempts to incorporate such assessments of ADCC with regard to the function of alemtuzumab were therefore abandoned.

Resident populations of Langerhans cells and dermal-interstitial dendritic cells do not express CD52⁺, similar to the progeny generated in vitro under cytokine-supported conditions

One of the hazards of drawing conclusions based on cells generated in vitro under the aegis of non-physiologic doses of recombinant cytokines is that the culture itself may have artifactually skewed the results. Therefore CD52⁺ protein expression was examined immunohistochemically on archival human tissue (FIG. 6). The specimens included two samples each of normal skin and intestinal tissue, skin biopsies from patients with drug hypersensitivity reactions, and skin biopsies from patients with GvHD. None of the biopsies showed co-expression of S-100 protein-positive cells with CD52⁺. Thus, resident epidermal or mucosal Langerhans cells (LCs) were immunonegative for CD52⁺. Likewise, coexpression of CD52⁺ and S-100-protein-positive inflammatory cells of the dermis never occurred. Hence, dermal dendritic cells (DDC-IDCs) were also immunonegative for CD52⁺. These results argue definitively against the possibility that exogenous cytokines in vitro could have modulated CD52⁺ surface expression compared with that found on resident populations of DCs in vivo.

Discussion

The successful application of anti-CD52⁺ in clinical transplantation derives in large part from its depletion of alloreactive T cells. This mechanism supports recent reports that recombinant DNA-derived, humanized anti-CD52⁺ (alemtuzumab, CAMPATH-1H), administered in vivo, reduces the incidence and severity of GvHD after allogeneic HSCT while preserving the graft-versus-tumor benefit of the allograft, although follow-up is admittedly still limited (11,12). Investigators have also now shown that anti-CD52⁺ MAb (CAMPATH-1G) eliminates host moDCs, compared with standard chemotherapy and radiation-based preparative regimens; but does not impair recovery of donor-derived moDCs(3).

It was asked whether, in addition to T lymphocytes, distinct types of dendritic cells differentially expressed CD52⁺ for targeting by alemtuzumab. Using three carefully defined populations of myeloid DCs, it was found that moDCs expressed CD52⁺ to the exclusion of other myeloid DC populations. The most surprising and novel finding was that LCs and DDC-IDCs were in fact always negative for the CD52⁺ epitope, either as resident populations in normal or inflamed skin or gut, or as cytokine-generated progeny of CD34+ HPCs in vitro. Although the operative mechanism by which humanized anti-CD52⁺ (alemtuzumab) exerts its effects in vivo is likely by ADCC⁶, MAb/complement-dependent lysis-provided a useful approximation of anti-CD52⁺ function for our,studies in vitro.

Alemtuzumab, or anti-CD52⁺, caused complement-dependent lysis in proportion to the amount of surface CD52⁺ expression and was therefore lytic only for moDCs but not for either immature or mature LCs or DDC-IDCs. CD34+ HPCs also proved resistant, perhaps reflecting their lower surface density of CD52⁺ expression. This resistance, however, is also consistent with the well documented preservation of transplantable stem cells after anti-CD52⁺ purging in vitro (33,34). It may also account for the absence of actual reported cases of aplasia from treatment with alemtuzumab in vivo.

CD52⁺ exerted no adhesive or proliferative functions between DCs and T cells, because anti-CD52⁺ alone in the absence of complement inhibited neither DC:T cell aggregation nor alloantigen-specific T cell proliferative responses. CD52⁺ also had no effect on moDC development, as binding of CD52⁺ by the MAb did not alter cytokine-driven differentiation in vitro.

The fact that anti-CD52⁺ mAb therapy with alemtuzumab does not result in increased GvHD, given the persistence of LCs and DDC-IDCs, suggests two possible explanations. One is that the dominant effect of alemtuzumab is to cause such profound T cell depletion that survival of LCs and DDC-IDCs is irrelevant. Our data suggest an additional possibility, which is that elimination of moDCs from the inflammatory environment early post-transplant removes a highly phagocytic and potent APC that could otherwise present Ag from dying host cells to surviving or newly generated donor T cells.

Several lines of evidence are offered in support of this reasoning. First of all, despite the absence of randomized trials, comparable levels of T cell depletion by other transplant regimens have not regularly achieved success similar to anti-CD52⁺-containing regimens in terms of reduced GvHD and immune reconstitution, especially in older adults receiving unrelated and/or mismatched allografts. This is true even when G-CSF-elicited, T cell-depleted, peripheral blood stem cells containing disproportionate numbers of Th2-inducing DCs (35) have been used as the source of the allograft. Alemtuzumab may therefore mediate either qualitative differences in T cell depletion or differentially affect antigen-presentation in contrast to other T cell depletion approaches. Secondly, infections by viruses like cytomegalovirus (CMV) seem not to be exerting negative effects on patient survival after alemtuzumab-facilitated transplants, to the same extent as comparable CMV reactivation rates after other methods of T cell depletion¹¹, suggesting more effective immune reconstitution. Although follow-up is still limited, relapse is so far not a greater cause of treatment failure after preparative regimens using anti-CD52⁺ (11,12). It would therefore be proposed that preservation of LCs and DDC-IDCs after anti-CD52⁺ treatment allows these myeloid DCs play formative roles in the redevelopment of acquired and beneficial immunity.

Several lines of evidence support that moDCs are prime candidates for eliciting GvHD reactions, at least acutely. Monocytes and monocyte-derived DCs are intensely phagocytic (36), especially in comparison to other myeloid DCs, like Langerhans cells and DDC/IDCs (Ratzinger et al., in preparation). In the inflammatory environment of an allogeneic transplant, circulating monocyte precursors and immature moDCs would be ideally suited for uptake of dying host cells. Differentiation into mature moDCs and presentation of these host antigens to-reactive clones of T cells circulating through secondary lymphoid organs would follow. Recent data from Ferrara and colleagues indicate that LPS and CD14 are critical to the induction of experimental acute GvHD (37,38), further implicating a specific role for CD14+ monocyte-derived DCs in the sensitization,arm of this process.

In the case of hematopoietic stem cell allografts, monocyte-derived DCs of either host (39) or donor origin could phagocytose dying host cellular Ags for presentation to and sensitization of engrafting donor-derived T cells. These would in turn cause GvHD in the periphery, especially in those sites that most abundantly express the same MHC antigens, e.g., skin, gut, liver, and lymphoid organs. In the case of other nonmyeloablative preparative regimens that allow persistence of host T cells, donor monocyte-derived DCs could even sensitize host T cells by the direct pathway, leading to host resistance and nonengraftment or rejection.

Similar logic applies to solid organ allografts, where MHC disparities are the rule rather than the exception and where chronic rejection and long term graft survival remain problematic. Donor monocyte-derived DCs transferred among the so-called passenger leukocytes in an allograft could directly sensitize host T cells. Alternatively, host monocyte-derived DCs could phagocytose and present donor-derived cellular antigens from dying cells in the inflammatory environment of the transplanted allograft. Humanized anti-CD52⁺ therapy should alter or eliminate both processes.

These results have important implications for the activity of alemtuzumab in allogeneic transplantation. Like other studies in this area (40-42), our findings support greater attention to the afferent arm of the immune response, rather than focusing on alloreactive T cell responders/effectors to the potential, exclusion of evaluating antigen presentation. This leads us to hypothesize that the undesirable complications of GvHD or rejection (host versus graft) may be distinguishable from the beneficial graft-versus-leukemia or graft-versus-tumor effects exerted by hematopoietic allografts, based on differences in afferent sensitization of an immune response by different types of myeloid DCs.

If true, this would predict that the selective elimination of monocyte-derived DCs by anti-CD52⁺ in the inflammatory environment early after transplantation may promote the long term tolerance and graft survival that has been so difficult to achieve with only T cell targeted immune suppression in mismatched and/or unrelated host-donor pairings. Accordingly, preservation of resident LCs and DDC-IDCs may be as important to generating GvT and reconstituting normal cellular immunity, as is depletion of highly phagocytic moDCs to the reduction of GvHD in the early and highly inflammatory post-transplant environment. This does not exclude a role, however, for moDCs in the generation and maintenance of peripheral tolerance or GvT at later time points when the inflammatory cytokine milieu is substantially diminished. It also does not exclude a more dominant effect of anti-CD52⁺ on T cell responder populations, regardless of the DC populations that may be left intact or not. These concepts merit further specific testing in preclinical animal models and clinical trials.

### TABLE 1

Phenotypic Expression of CD52⁺

Cells were stained and analyzed by flow cytometry as described. The cell types were gated for their respective phenotypes, and the percent of those cells expressing CD52⁺ was determined. Mean fluorescent index (MFI) of the CD52⁺ positive cells was also calculated by the CellQuest software on the FACScan as a measure of CD52⁺ epitope density on the cell surface. T cells were total CD4 and CD8 positive lymphocytes. Monocytes were CD14+ PBMCs. All DC populations were large FSC and HLA-DR bright cells. Immature DCs were CD83 neg, whereas mature DCs were: CD83+ and additionally increased expression of CD40, CD80, and CD86. LCs were CD11b neg, whereas moDCs and DDCs were CD11b:positive. MoDCs were differentiated from CD14+ monocytes as described, and CD34+ hematopoietic progenitor cells (HPCs) generated LCs and DDCs. DDCs nominally included both dermal (DDC) and interstitial (IDC) DCs. Fresh circulating DCs were defined as CD11c+, lineage-negative (CD 3, CD14, CD16, CD20-negative) PBMCs.

### TABLE 2

Assessment of anti-CD52⁺ on the Development of moDCs in Vitro

In vitro cultures for the generation of moDC from CD14+ monocytes were supplemented with humanized anti-CD52⁺ (alemtuzumab, CAMPATH-1H) or anti-CD20 (rituximab) as a negative control, both at maximal concentrations of 1 mg/ml. Viable cell numbers were based on trypan blue exclusion by direct hemacytometer counts and were expressed as a percentage relative to the starting number of monocytes at day 0. Annexin V and propidium iodide staining to identify the percent apoptotic cells in culture did not reveal any substantial differences relative to the negative control MAb. Staining for CD83, as well as CD40, CD80, and CD86 (not shown) confirmed efficient maturation in both conditions. Proliferation of responder T cells in allogeneic mixed leukocyte reaction showed equal stipulatory capacity for moDC generated in the presence of CAMPATH-1H compared to the negative control.

TABLE 1
Phenotypic expression of CD52⁺

| | | CD14+ | immature | mature | CD3+ | immature | mature | Immature | mature | ciculating |
|---|---|---|---|---|---|---|---|---|---|---|
| | T cells | monocytes | moDCs | moDCs | HPCs | LCs | LCs | DDCs | DDCs | blood DCs |
| % cells positive | 100 | 100 | 98 | 95 | 84 | 1 | 5 | 2 | 2 | 100 |
| mean fluorescent | 1808 | 310 | 105 | 56 | 50 | 6 | 5 | 7 | 7 | 1862 |
| intensity | | | | | | | | | | |
| (MFI) | | | | | | | | | | |

TABLE 2
Assessment of anti-CD52⁺on the development of moDCs in vitro

| | Cell number | Cell number | % | % | % | Stimulatory |
|---|---|---|---|---|---|---|
| | Day 6 | Day 8 | apoptotic cells | apoptotic cells | CD83+ cells | capacity |
| | relative to d0 | Relative to d0 | Day 6 | Day 8 | Day 8 | Allo MLR |
| Anti-CD52⁺ | 114% | 108% | 5% | 10% | 94% | 99% |
| (alemtuzumb, | | | | | | |
| CAMPATH-1H) | | | | | | |
| Anti-CD20 | 100% | 100% | 8% | 20% | 92% | 100% |
| negative control | | | | | | |
| (rituximab) | | | | | | |

### REFERENCES

1. Xia M Q, Tone M, Packman L, Hale G, Waldmann H. Characterization of the CAMPATH-1 (CDw52) antigen: biochemical analysis and cDNA cloning reveal an unusually small peptide backbone. Eur J Immunol. 1991;21:1677-1684.
2. Hart D, Fearnley D, Sorg U, Hock B: Non-lineage antigens and dendritic cells functional studies: the CMRF-56 monoclonal antibody identifies dendritic cells after brief culture of human peripheral blood mononuclear cells. In Kishimoto T, Kikutani H, von dem Borne E G K, Goyert S M, Mason D Y, Miyasaka M, Moretta L, Okumura K, Shaw S, Springer T A, Sugamura K, Zola H (eds). Leukocyte Typing VI (ed 6th). New York, N.Y.; Garland Publishing, Inc.; 1997:599-601.
3. Klangsinsirikul P, Carter G I, Byrne J L, Hale G, Russell N H. CAMPATH-1G causes rapid depletion of circulating host dendritic cells (DCs) before allogeneic transplantation but does not delay donor DC reconstitution. Blood. 2002; 99:2586-2591.
4. Rowan W C, Hale G, Tite J P, Brett S J. Cross-linking of the CAMPATH-1 antigen (CD52⁺) triggers activation of normal human T lymphocytes. Int Immunol. 1995;7:69-77.
5. Xia M Q, Hale G, Waldmann H. Efficient complement-mediated lysis of cells containing the CAMPATH-1 (CDw52) antigen. Mol Immunol. 1993;30:1089-1096.
6. Dyer M, Hale G, Hayhoe F, Waldmann H. Effects of CAMPATH-1 antibodies in vivo in patients with lymphoid malignancies: influence of antibody isotype. Blood. 1989;73:1431-1439.
7. Isaacs J D, Wing M G, Greenwood J L D, Hazelman B L, Hale G, Waldmann H. A therapeutic human IgG4 monoclonal antibody that depletes target cells in humans. Clin Exp Immunol. 1996;106:427-433.
8. Riechmann L, Clark M, Waldmann H, Winter G. Reshaping human antibodies for therapy. Nature. 1988;332:323-327.
9. Rebello P, Hale G. Pharmacokinetics of CAMPATH-1H: assay development and validation. J Immunol Methods. 2002;260:285-302.
10. Flynn J M, Byrd J C. CAMPATH-1H monoclonal antibody therapy. Curr Opin Oncol. 2000;12:574-581.
11. Chakraverty R, Peggs K, Chopra R, et al. Limiting transplantation-related mortality following unrelated donor stem cell transplantation by using a nonmyeloablative conditioning regimen. Blood. 2002;99:1071-1078.
12. Kottaridis P D, Milligan D W, Chopra R, et al. In vivo CAMPATH-1H prevents graft-versus-host disease following nonmyeloablative stem cell transplantation. Blood. 2000;96:2419-2425.
13. Banchereau J, Steinman R M. Dendritic cells and the control of immunity. Nature. 1998;392:245-252.
14. Bell D,. Young J W, Banchereau J. Dendritic cells. Adv. Immunol. 1999;72:255-322.
15. Hart D N J. Dendritic cells: Unique leukocyte populations which control the primary immune response. Blood. 1997;90:3245-3287.
16. Caux C, Massacrier C, Vanbervliet B, et al. CD34+ hematopoietic progenitors from human cord blood differentiate along two independent dendritic cell pathways in response to granulocyte-macrophage colony-stimulating factor plus tumor necrosis factor. Blood. 1997;90:1458-1470.
17. Caux C, Vanbervliet B, Massacrier C, et al. CD34+ hematopoietic progenitors from human cord blood differentiate along two independent dendritic cell pathways in response to GM-CSF+TNF alpha. J. Exp. Med. 1996;184:695-706.
18. Caux C, Dezutter-Dambuyant C, Schmitt D, Banchereau J. GM-CSF and TNF-a cooperate in the generation of dendritic Langerhans cells. Nature. 1992;360:258-261.
19. Szabolcs P, Moore M A S, Young J W. Expansion of immunostimulatory dendritic cells among the myeloid progeny of human CD34+ bone marrow precursors cultured with c-kit ligand, granulocyte-macrophage colony-stimulating factor, and TNF-a. J. Immunol. 1995;154:5851-5861.
20. Szabolcs P, Avigan D, Gezelter S, et al. Dendritic cells and macrophages can mature independently from a human bone marrow-derived, post-CFU intermediate. Blood. 1996;87:4520-4530.
21. Young J W, Szabolcs P, Moore M A S. Identification of dendritic cell colonyforming units among normal CD34+ bone marrow progenitors that are expanded by c-kit-ligand and yield pure-dendritic cell colonies in the presence of granulocyte/macrophage colonystimulating factor and tumor necrosis factor a. J. Exp. Med. 1995;182:1111-1120.
22. Strobl H, Riedl E, Scheinecker C, et al. TGF-b1 promotes in vitro development of dendritic cells from CD34+ hemopoietic progenitors. J. Immunol. 1996;157:1499-1507.
23. Gatti E, Velleca M A, Biedermann B C, et al. Large-scale culture and selective maturation of human Langerhans cells from granulocyte colony-stimulating factor-mobilized CD34+ progenitors. J Immunol. 2000;164:3600-3607.
24. Romani N, Gruner S, Brang D, et al. Proliferating dendritic cell progenitors in human blood. J. Exp. Med. 1994;180:83-93.
25. Sallusto F, Lanzavecchia A. Efficient presentation of soluble antigen by cultured human dendritic cells is maintained by granulocyte/macrophage colony-stimulating factor plus interleukin 4 and downregulated by tumor necrosis factor a. J. Exp. Med. 1994;179:1109-1118.
26. Zhou L-J, Tedder T F. Human blood dendritic cells selectively express CD83, a member of the immunoglobulin superfamily. J. Immunol. 1995;154:3821-3835.
27. Bender A, Sapp M, Schuler G, Steinman R M, Bhardwaj N. Improved methods for the generation of dendritic cells from nonproliferating progenitors in human blood. J. Immunol. Methods. 1996;196:121-135.
28. Thurner B, Roder C, Dieckmann D, et al. Generation of large numbers of fully mature and stable dendritic cells from leukapheresis products for clinical application. J. Immunol. Meth. 1999;223:1-15.
29. Rieser C, Bock G, Klocker H, Bartsch G. Thurnher M. Prostaglandin E2 and tumor necrosis factor a cooperate to activate human dendritic cells: Synergistic activation of interleukin 12 production. J. Exp. Med. <1997; 186:1603-1608.
30. -Jonuleit H, Kuhn U, Muller G, et al. Pro-inflammatory cytokines and prostaglandins induce maturation of potent immunostimulatory dendritic cells under fetal calf serum-free conditions. Eur. J. Immunol. 1997;27:3135-3142.
31. Jansen J H, Wientjens G-J H M, Fibbe W E, Willemze R. Kluin-Nelemans H C. Inhibition of human macrophage colony formation by interleukin 4. J. Exp. Med. 1989;170:577-582.
32. Weiner L M. Monoclonal antibody therapy of cancer. Semin Oncol. 1999;26:43-51.
33. Gilleece M, Dexter T. Effect of CAMPATH-1H antibody on human hematopoietic progenitors in vitro. Blood. 1993;82:807-812.
34. Gerritsen W R, Wagemaker G, Jonker M, et al. The repopulation capacity of bone marrow grafts following pretreatment with monoclonal antibodies against T lymphocytes in rhesus monkeys. Transplantation. 1988;45:301-307.
35. Arpinati M, Green C L, Heimfeld S, Heuser J E, Anasetti C. Granulocyte-colony stimulating factor mobilizes T helper 2-inducing dendritic cells. Blood. 2000;95:2484-2490.
36. Albert M L, Sauter B, Bhardwaj N. Dendritic cells acquire antigen from apoptotic cells and induce class I-restricted CTLs. Nature. 1998;392:86-89.
37. Cooke K R, Gerbitz A, Crawford J M, et al. LPS antagonism reduces graft-versushost disease and preserves graft-versus-leukemia activity after experimental bone marrow transplantation. J Clin Invest. 2001;107:1581-1589.
38. Cooke K, Olkiewicz K, Clouthier S, Liu C, Ferrara J: Critical role for CD14 and the innate immune response in the induction of experimental acute graft-versus-host disease. American Society of Hematology 43rd Annual Meeting and Exposition. Orlando, Fla., 2001, p 776a.
39. Shlomchik W D, Couzens M S, Tang C B, et al. Prevention of graft versus host disease,by inactivation of host antigen-presenting cells. Science. 1999;285:412-415.
40. Hill G R, Ferrara J L M. The primacy of the gastrointestinal tract as a target organ of acute graft-versus-host disease: rationale for the use of cytokine shields in allogeneic bone marrow transplantation. Blood. 2000;95:2754-2759.
41. Ordemann R, Hutchinson R, Friedman J, et al. Enhanced allostimulatory activity of host antigen-presenting cells in old mice intensifies acute graft-versus-host disease. J. Clin. Invest. 2002;109:1249-1256.
42. Teshima T, Ordemann R, Reddy P, et al. Acute graft-versus-host disease does not require alloantigen expression on host epithelium. Nat Med. 2002;8:575-581.

1-19. cancel

20: An agent capable of destruction of CD52⁺ cells, including CD52⁺ dendritic cells, without affecting CD52⁺ negative dendritic cells.

21: An agent capable of selective elimination of CD52⁺ dendritic cells.

22: A composition comprising the agent of claim 20.

23: A composition comprising the agent of claim 21.

24: A pharmaceutical composition comprising an effective amount of the agent of claim 20 and a pharmaceutically acceptable carrier.

25: A pharmaceutical composition comprising an effective amount of the agent of claim 21 and a pharmaceutically acceptable carrier.

26: A pharmaceutical composition for prevention, treatment, or elimination of graft versus host disease comprising an effective amount of the agent of claim 20 and a pharmaceutically acceptable carrier.

27 A pharmaceutical composition for prevention, treatment, or elimination of graft versus host disease comprising an effective amount of the agent of claim 21 and a pharmaceutically acceptable carrier.

28: A pharmaceutical composition that promotes or maintains the graft versus tumor effect of hematopoietic stem cell transplantation, comprising an effective amount of the agent of claim 20 and a pharmaceutically acceptable carrier.

29: A pharmaceutical composition that promotes or maintains the graft versus tumor effect of hematopoietic stem cell transplantation, comprising an effective amount of the agent of claim 21 and a pharmaceutically acceptable carrier.

30: The agent of claim 20, which binds to CD52⁺ cells

31: The agent of claim 21, which binds to CD52⁺ cells.

32: The agent of claim 30, which is an antibody or a portion thereof.

33: The agent of claim 31, which is an antibody or a portion thereof.

34: The agent of claim 30, which comprises an anti-sense nucleic acid molecule.

35: The agent of claim 31, which comprises an anti-sense nucleic acid molecule.

36: The agent of claim 30, further comprising a toxin.

37: The agent of claim 31, further comprising a toxin.

38: A method for preventing or treating graft versus host disease in a subject comprising selective elimination of CD52⁺ dendritic cells in the subject without affecting CD52⁺ negative dendritic cells.

39: A method for preventing or treating graft versus host disease in a subject comprising administration of an effective amount of the agent of claim 20 to the subject.
